(19) United States
(12) Patent Application Publication
(10) Pub. No.: US 2025/0334572 A1
(43) Pub. Date: Oct. 30, 2025

(54) HOST CELL PROTEIN DETECTION USING LATERAL FLOW DEVICES

(71) Applicant: Waters Technologies Corporation, Milford, MA (US)

(72) Inventors: Anna Boardman, Watertown, MA (US); Jianmin Liu, Northborough, MA (US); Lingyun Chen, Westborough, MA (US); Kevin Wyndham, Worcester, MA (US); Lindsay Morrison, Waltham, MA (US)

(73) Assignee: Waters Technologies Corporation, Milford, MA (US)

(21) Appl. No.: 19/185,718

(22) Filed: Apr. 22, 2025

Related U.S. Application Data
(60) Provisional application No. 63/639,330, filed on Apr. 26, 2024.

(51) Int. Cl. G01N 33/543; G01N 33/569; G01N 33/68

(52) U.S. Cl. CPC G01N 33/54388; G01N 33/56916; G01N 33/56938; G01N 33/56961; G01N 33/56966; G01N 33/6803; G01N 2333/21; G01N 2333/245; G01N 2333/31; G01N 2333/395; G01N 2333/46

(57) ABSTRACT

Time sensitive biopharmaceutical processing updates can be implemented using lateral flow devices to test on-site during manufacturing. Lateral flow testing for an analyte such as, for example, impurities (e.g., host cell proteins), during manufacturing of a biopharmaceutical drug product is completed within twenty-five minutes of collecting a sample from the manufacturing line. That is, all sample preparation, such as adding reagent or diluting the sample, contacting the prepared sample to a lateral flow device, and detecting presence or absence of the analyte is completed within twenty-five minutes (e.g., 20 minutes, 18 minutes, 15 minutes) from collection such that processing adjustments during manufacturing can be implemented.

### CROSS REFERENCE TO RELATED APPLICATIONS

This application claims the benefit of and priority to U.S. provisional patent application Ser. No. 63/639,330 entitled “Host Cell Protein Detection Using Lateral Flow Devices”, the content of which are hereby incorporated by reference in its entirety.

### TECHNICAL FIELD

The present disclosure relates generally to devices and methods for lateral flow testing. In particular, the present disclosure relates to devices and methods for achieving timely and reliable information during production of biopharmaceuticals using lateral flow devices.

### BACKGROUND

Host cell proteins (HCPs) are process-related protein impurities generated by host organisms during the production of biopharmaceuticals. HCPs are a complex mixture of various proteins with diverse physiochemical properties which can impact drug efficacy and cause immunogenic responses in patients. Oftentimes it is necessary to minimize HCP presence by various means including optimizing the cell culture conditions, modifying the purification process, or using additional purification or filtering steps to remove the HCPs during production of biopharmaceuticals, to limit the amount present in a final product delivered to a patient. HCP analysis is performed at multiple stages during biopharmaceutical development, including cell culture formation, purification, and final product testing. Additionally, regulatory agencies (i.e., FDA) require HCPs to be measured by sponsors to ensure quality of the final drug product for clinical and commercial use. Current guidelines require less than 100 ppm (e.g., less than 100 ng/mg) of HCPs in a drug product.

To test for HCPs, conventional methods utilize ELISA (enzyme linked immunosorbent assay), Western blot, or LC-MS assay techniques. While these techniques provide accurate results, the time scale and testing requirements are burdensome. ELISA and Western blot testing require more than an hour of analysis time, and all three techniques requiring additional sample preparation time conducted in a controlled environment (i.e., dedicated laboratory). As a result of these requirements, testing often and in a timescale needed to optimize or change processing parameters of a biopharmaceutical is unavailable.

### SUMMARY

The present technology utilizes lateral flow devices to test during the production of biopharmaceutical drug products on a timescale and accuracy to optimize or change processing conditions to achieve a desired drug product. For example, lateral flow testing can be implemented during production to reduce the presence of impurities (e.g., host cell proteins) numerous times during one or more of formation of a cell culture, purification, and final product testing.

Lateral flow testing can be used to assess concentrations of an analyte in solution. The use of lateral flow devices became commonplace during the COVID-19 pandemic as these devices are easy to use and require very little sample preparation or user expertise to perform. The robustness of lateral flow testing devices allows for more active testing of products at the source, such as during production of a cell line, downstream processing such as filtration or purification, or any other production step. In the present technology, lateral flow devices are used to detect the presence or absence (or even an amount) of HCPs in under 30 minutes (e.g., 25 minutes, 20 minutes, 18 minutes, 16 minutes) from obtaining a sample from a reactor or other downstream component of the biopharmaceutical manufacturing process. That is, the detected result is obtained on-site, in a timeframe that allows for optimization of the biopharmaceutical manufacturing process.

In one aspect, the present technology relates to a method of detecting presence of host cell proteins during production of a biopharmaceutical. The method according to this aspect of the technology includes a) removing a liquid sample from a biopharmaceutical production reactor; b) contacting the liquid sample to a lateral flow device, wherein the lateral flow device comprises a nitrocellulose membrane and a region of polyclonal antibodies that specifically bind to host cell proteins from a host cell line or a host cell culture; and c) detecting presence or absence of more than a threshold concentration of host cell proteins within twenty-five minutes from removing the liquid sample from the biopharmaceutical production reactor, wherein the threshold concentration of host cell proteins is 100 ppm.

In another aspect, the present technology relates to a method of detecting presence of host cell proteins during production of a biopharmaceutical. The method according to this aspect of the technology includes: a) removing a liquid sample from a biopharmaceutical production reactor; b) contacting the liquid sample to a lateral flow device, wherein the lateral flow device comprises a nitrocellulose membrane and a region of proteins or peptides from a host cell line or a host cell culture; and c) detecting presence or absence of more than a threshold concentration of host cell proteins within the liquid sample within twenty-five minutes from removing the liquid sample from the biopharmaceutical production reactor, wherein the threshold concentration of host cell proteins is 100 ppm.

The methods according to any of the above aspects can include one or more of the following features. Some embodiments feature a lower threshold concentration than 100 ppm. In some embodiments, the threshold concentration of host cell proteins is 50 ppm, 10 ppm, or 1 ppm. In some embodiments, the step of “detecting presence or absence of more than the threshold concentration of host cell proteins” is within twenty minutes (e.g., 20 minutes, 19 minutes, 18 minutes, 16 minutes, etc.) from removing the liquid sample from the biopharmaceutical production reactor. In some embodiments, the step of “detecting presence or absence of more than the threshold concentration of host cell proteins” is within fifteen minutes (e.g., 15 minutes, 14 minutes) from removing the liquid sample from the biopharmaceutical production reactor.

Embodiments of the above aspects can feature sample preparation. For example, the liquid sample from the biopharmaceutical production reactor, in some embodiments, is combined with a reagent prior to contacting the liquid sample to the lateral flow device. The reagent can include a buffer and gold particles conjugated to a polyclonal antibody of the host cell line or host cell culture. In some embodiments the reagent includes a diluent. In some embodiments, the liquid sample from the biopharmaceutical production reactor is filtered prior to contacting the liquid sample to the lateral flow device. In certain embodiments, the liquid sample from the biopharmaceutical production reactor is diluted prior to contacting the liquid sample to the lateral flow device.

Some embodiments of the above aspects feature Chinese hamster ovary as the host cell line. In other embodiments, the host cell line is selected from the group consisting of HEK-293, HeLa, MDCK, A549, ad Sf9. In some embodiments, the host cell culture is selected from the group consisting of Escherichia coli, Lactococcus lactis, Pseudomonas fluorescens, Staphylococcus aureus, Saccharomyces cerevisiae, Pichia pastoris (also known as Komagataella phaffii).

Some embodiments of the above aspects feature a control line on the lateral flow device. The methods featuring a control line can include one or more of the following features. Some methods further include quantifying the concentration of host cell proteins present in the liquid sample via evaluating a light absorption level for a test line within the region of polyclonal antibodies and a light absorption control line level. Some methods further include quantifying the concentration of host cell proteins present in the liquid sample via evaluating a light absorption level for a test line within the region of proteins or peptides and a light absorption control line level. In some embodiments, the method further includes measuring a value for the light absorption level of the test line and a control value for the light absorption control line level, and determining a test to control ratio. Certain embodiments feature inserting at least a portion of the lateral flow device into a reader device for measuring a value of light absorption for the test line and the control line. The lateral flow device can include a housing, which has a transparent window for reading the control line and the test line. The housing can further include a calibration device affixed to the housing.

The liquid sample from the biopharmaceutical production reactor can be sampled one or more times. In some embodiments of the above aspects, the liquid sample is removed during a cell culturing step during production of the biopharmaceutical. In some embodiments, the liquid sample is removed from the biopharmaceutical production reactor prior to a purification of a cell culture within the biopharmaceutical production reactor. In some embodiments, the liquid sample is removed from the biopharmaceutical production reactor during a purification of a cell culture within the biopharmaceutical production reactor. In some embodiments, the liquid sample is removed from the biopharmaceutical production reactor after a purification of a cell culture within the biopharmaceutical production reactor. Some embodiments feature removing a second liquid sample from the biopharmaceutical production reactor and contacting the second liquid sample to a second lateral flow device at a later stage of processing. Certain embodiments feature removing a plurality of liquid samples from the biopharmaceutical production and detecting presence or absence of host cell proteins in each of the plurality of liquid samples using a dedicated lateral flow device.

In one aspect, the present technology relates to a method of detecting presence of host cell proteins during production of a biopharmaceutical. The method according to this aspect of the technology includes a) collecting a sample from a biopharmaceutical drug product manufacturing line in a vial; b) mixing the sample with a reagent to form a prepared sample; c) contacting the prepared sample to a lateral flow device, wherein the lateral flow device comprises a nitrocellulose membrane and a region of polyclonal antibodies that specifically bind to host cell proteins from a host cell line or a host cell culture; and d) detecting presence or absence of more than a threshold concentration of host cell proteins within twenty-five minutes from collecting the sample from the biopharmaceutical drug product manufacturing line, wherein the threshold concentration of host cell proteins is 100 ppm.

In another aspect, the present technology relates to a method of detecting presence of host cell proteins during production of a biopharmaceutical. The method according to this aspect of the technology includes a) collecting a sample from a biopharmaceutical drug product manufacturing line in a vial; b) mixing the sample with a reagent to form a prepared sample; c) contacting the prepared sample to a lateral flow device, wherein the lateral flow device comprises a nitrocellulose membrane and a region of proteins or peptides from a host cell line or a host cell culture; and d) detecting presence or absence of more than a threshold concentration of host cell proteins within the liquid sample within twenty-five minutes from collecting the sample from the biopharmaceutical drug product manufacturing line, wherein the threshold concentration of host cell proteins is 100 ppm.

The methods according to any of the above aspects can include one or more of the following features. Some embodiments feature a lower threshold concentration than 100 ppm. In some embodiments, the threshold concentration of host cell proteins is 50 ppm, 10 ppm, or 1 ppm. In some embodiments, the step of “detecting presence or absence of more than the threshold concentration of host cell proteins” is within twenty minutes (e.g., 20 minutes, 19 minutes, 18 minutes, 16 minutes, etc.) from collecting the liquid sample from the biopharmaceutical drug product manufacturing line. In some embodiments, the step of “detecting presence or absence of more than the threshold concentration of host cell proteins” is within fifteen minutes (e.g., 15 minutes, 14 minutes) from collecting the liquid sample from the biopharmaceutical drug product manufacturing line.

Embodiments of the above aspects can feature collecting the samples at one or more times during production. For example, some methods include collecting the sample from a biopharmaceutical drug product manufacturing line occurs at cell harvest. Some methods include collecting the sample from a biopharmaceutical drug product manufacturing line occurs prior to a filtration step. Certain methods include collecting the sample from a biopharmaceutical drug product manufacturing line occurs after a filtration step. Some methods include collecting the sample from a biopharmaceutical drug product manufacturing line occurs during an affinity capture step. Certain methods include collecting the sample from a biopharmaceutical drug product manufacturing line occurs after a buffer exchange step. Some embodiments feature collecting a second sample from the biopharmaceutical drug product manufacturing line, mixing with a second reagent and contacting to a second lateral flow device at different stage of drug product manufacturing. Certain embodiments feature collecting a plurality of samples from the biopharmaceutical drug product manufacturing line, mixing with a dedicated reagent, and determining presence or absence of host cell proteins in each of the plurality of samples using a dedicated lateral flow device.

Methods of the present disclosure provide several advantages over the prior art. For example, the devices and methods of the present disclosure can provide accurate results on the present or absence (e.g., 100 ppm sensitivity) of host cell proteins in a sample within 25 minutes of collection. The present technology utilizes lateral flow devices which can be conducted by manufacturing line personnel. That is, testing using lateral flow devices is conducted on-site during manufacturing. The results are available within thirty minutes of collecting a sample and thus allow for adjustment or optimization of the manufacturing process based on the results from the lateral flow test. Additional advantages include the ability to conduct lateral flow tests at one or more different time points during the manufacturing process. In addition, a test reader can be used in connection with the lateral flow strips to provide semi-quantitative or quantitative results.

### BRIEF DESCRIPTION OF THE DRAWINGS

The technology will be more fully understood from the following detailed description taken in conjunction with the accompanying drawings, in which:

FIG. 1 provides a flow chart illustrating a method in accordance with an embodiment of the present technology.

FIG. 2A and FIG. 2B illustrate an embodiment (competitive format) of a lateral flow device in accordance with the present technology. FIG. 2A provides a schematic showing a lateral flow test (competitive format) after testing a sample which lacks host cell proteins (i.e., show the absence of HCPs) in the tested sample; whereas FIG. 2B provides a schematic showing a lateral flow test (competitive format) after testing a sample which includes at least a threshold amount (i.e., a detectable amount) of host cell proteins.

FIG. 2C shows the results of a test on a competitive format lateral flow device for a sample that was doped with a 1 parts per thousand (ppt) concentration of HCPs; and a second lateral flow device for a sample that was known to be free of HCPs.

FIG. 3A and FIG. 3B illustrate an embodiment (sandwich format) of a lateral flow device in accordance with the present technology. FIG. 3A provides a schematic showing a lateral flow test (sandwich format) after testing a sample which lacks host cell proteins (i.e., show the absence of HCPs) in the tested sample; whereas FIG. 3B provides a schematic showing a lateral flow test (sandwich format) after testing a sample which includes at least a threshold amount (i.e., a detectable amount) of host cell proteins.

FIG. 4A shows a graph of Test Line to Control Line (T/C) responses for various known concentrations of host cell proteins (Chinese Hamster Ovary lysate); FIG. 4B shows an image of a series of lateral flow devices used to test samples with various known concentrations.

The features and advantages of the present disclosure will become more apparent from the detailed description set forth below when taken in conjunction with the drawings.

### DETAILED DESCRIPTION

### Definitions

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)₂, Fab, Fv, and scFv fragments.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “conjugated” refers to the linkage of two molecules formed by the chemical bonding of a reactive functional group of one molecule, such as a functionalized gold particle, with an appropriately reactive functional group of another molecule, such as a polyclonal antibody. See for example “Tools to compare antibody gold nanoparticle conjugates for a small molecule immunoassay” by Conrad et al. Mikrochim Acta 2023: 190(2)62, published on line 2023 Jan. 20. doi: 10.1007/s000604-023-05637-x.

As used herein, the term “non-specific binding” refers to the binding of unintended compounds to the antibody or antigen-binding fragment thereof.

As used herein, the term “specifically bind” refers to the intended binding of compounds to an antibody or antigen-binding fragment thereof.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein the term “biopharmaceutical” is a pharmaceutical or therapy derived from biological sources.

As used herein the term “host cell line” refers to eukaryotic cell line is used in the manufacture of a biopharmaceutical.

As used herein the term “host cell culture” refers to a culture of a microorganism (e.g., a bacterial or fungal species) used in the manufacture of a biopharmaceutical.

As used herein the term “host cell proteins” and/or “host cell peptides” are process-related proteinaceous impurities present in the host cell culture or host cell line used during biopharmaceutical manufacturing and production.

As used herein the term “ppm” refers to parts per million, which is interpreted herein as ng/mg when applied to the concentration of host cell peptides.

Unless defined otherwise, all technical and scientific terms used herein have the same meaning as commonly understood by one of ordinary skill in the art to which this disclosure belongs.

Impurities can have a negative impact on the stability, safety, and efficacy of biopharmaceuticals, such as protein therapeutics. Even a small amount (1 ppm) can cause a significant and potentially life-threatening immunogenic reaction. As such, control and making time-sensitive adjustments to a manufacturing line is desirable during the biopharmaceutical manufacturing process—from cell line formation, to purification, to harvest, and filtration.

For example, host cell proteins are a type of product-related impurity to monitor throughout the manufacturing process. Host cell proteins (HCPs) co-extracted with a therapeutic protein can contain enzymes such as oxidases and lipases that break down proteins over time, affecting the stability of the drug product. Other host cell proteins and binding agents carried over from purification and filtration steps may lead to mis-formulation of the drug product outside the therapeutic window. As such, it is desirable to know to what extent HCPs are formed within the bioreactor and during downstream processing.

Conventional methods of monitoring HCPs and other impurities utilize ELISA, Western blot, and LC-MS assays to determine the amount of impurities. These techniques typically require trained personnel to conduct the test outside of the manufacturing plant location—using dedicated equipment. The period of time from collection of a sample to test results is at best a few hours, depending on how close the testing location is from the manufacturing plant. The conventional methods do not allow for adjustment to the manufacturing procedure in a desired time frame (e.g., under an hour or half-an hour from collection of a sample).

Devices and methods described herein utilize lateral flow devices for providing information on the presence or absence of impurities such as HCPs within a half an hour of sample collection. Lateral flow assays are widely used in food, environmental, and clinical diagnostics. As a rapid on-site screening tool, lateral flow assay differentiates from other sophisticated instrumental tests because it can be conducted in the field, instead of in a laboratory or other dedicated environment. The present technology provides devices and methods tailored for the rapid collection of data on impurities during the manufacturing process of biopharmaceuticals. The devices and methods can be performed in under a half-an-hour from collection of a sample from the manufacturing process (e.g., from bioreactor or downstream processing component). As the methods can be easily performed on-site, active testing (i.e., testing at multiple time periods throughout the manufacturing process) can be completed to gather information. The information on impurities can be used to make informed decisions such as changing manufacturing process parameters (e.g., temperature, processing time, etc.) and the incorporation of additional purification and/or filtering steps. Further, the present technology is not limited to one device format. Device results can provide a qualitive result (yes/no result), a semi-quantitative result (indicative a threshold cutoff amount of impurity) or can be used in conjunction with a reader (e.g., Vertu lateral flow reader commercially available from Vicam, Milford, MA) to provide a quantitative result.

Methods of the present technology utilize polyclonal antibodies that specifically bind to host cell proteins from a host cell line or a host cell culture. For example, if the host cell line or culture is Chinese hamster ovary (CHO), a CHO polyclonal antibody (CHO pAb) is used. In some device formats (i.e., competitive format), a reagent including CHO pAb together with a gold conjugate is combined with the sample prior to contact with the nitrocellulose membrane of the competitive lateral flow device. A test line region of the lateral flow device includes proteins or peptides from a host cell line or a host cell culture. If the sample is free of CHO proteins then, the CHO pAb connected to the gold conjugate of the reagent binds with the test line. If the sample includes CHO proteins, the gold particles within the reagent are bound to the CHO proteins in the sample and are less available for reaction with the test line. As a result, the light absorbance from the test line will be weaker when CHO proteins are present in the sample when tested with the competitive format lateral flow device.

Polyclonal antibodies are also used in embodiments featuring sandwich format lateral flow devices. Specifically, a CHO polyclonal antibody can be used to for the test line on a sandwich format device, and then another CHO polyclonal antibody together with a gold conjugate is used as a reagent for the sample. In this format, the test line appears brighter or more visible when CHO proteins are present in the sample.

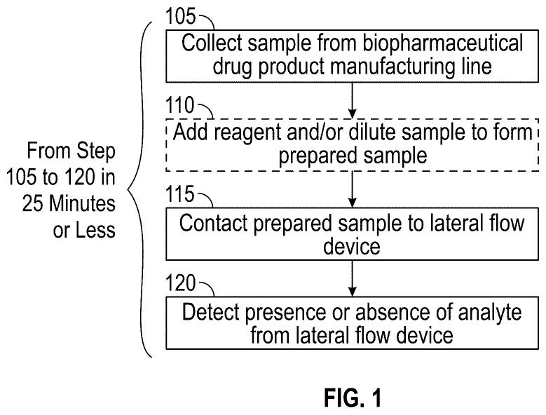

FIG. 1 illustrates a method of detecting presence of host cell proteins during production of a biopharmaceutical in accordance with an embodiment of the present technology. The method includes collecting a sample from a biopharmaceutical drug product manufacturing line (step 105); an optional sample preparation step (step 110); contacting the prepared sample to a lateral flow device (115); and detecting the presence or absence of an analyte (i.e., impurity, such as, host cell proteins) from the lateral flow device (120). It is notable that the entire method from collection (105) to detection (120) occurs in 25 minutes or less (e.g., 20 minutes, 18 minutes, 15 minutes).

While FIG. 1 illustrates an embodiment of the method, other embodiments are possible. For example, optional step 110 can further include dilution of the prepared sample. That is, in some embodiments, step 110 includes combining the sample with a reagent to form a prepared sample followed by dilution. In some embodiments, the diluent is added prior to combination with the reagent. The diluted prepared sample is then contacted to the lateral flow device in step 115. Even though multiple actions can be incorporated into step 110 of the method, the total period of time from collection 105 to detection 120 takes no more than 25 minutes. That is, any additional steps added to prepare the sample for contact with a lateral flow device 115 do not increase the time period from collection to detection to be greater than twenty-five minutes.

Due to the simplicity and timeliness of conducting of the technology, the method shown in FIG. 1 can be repeated multiple times using a dedicated lateral flow device throughout the manufacturing process. For example, the sample collected in step 105 can be from the bioreactor during the creation of the cell line or cell culture for the drug product. That is, the sample can be collected from the reactor one or more times during creation of the cells. Data regarding the presence of HCPs can be utilized to adjust the parameters of the reactor and or can be used for the incorporation of additional purification or filtration steps downstream of the reactor during the manufacturing process.

In addition to or alternatively, samples can be collected for step 105 of the method illustrated in FIG. 1 from other components during the manufacturing process. Specifically, one or more samples can be collected at cell harvest, prior to or after a filtration step, during or after an affinity capture step, or after a buffer exchange step. Multiple samples can be collected during any stage of manufacturing—and samples at different stages of the same manufacturing process can be collected and analyzed in accordance with the method illustrated in FIG. 1. Each sample is collected individually and is tested with a dedicated lateral flow device. The time period from each collection event to its corresponding detection event is twenty-five minutes or less (e.g., 20 minutes, 18 minutes, 15 minutes, etc.).

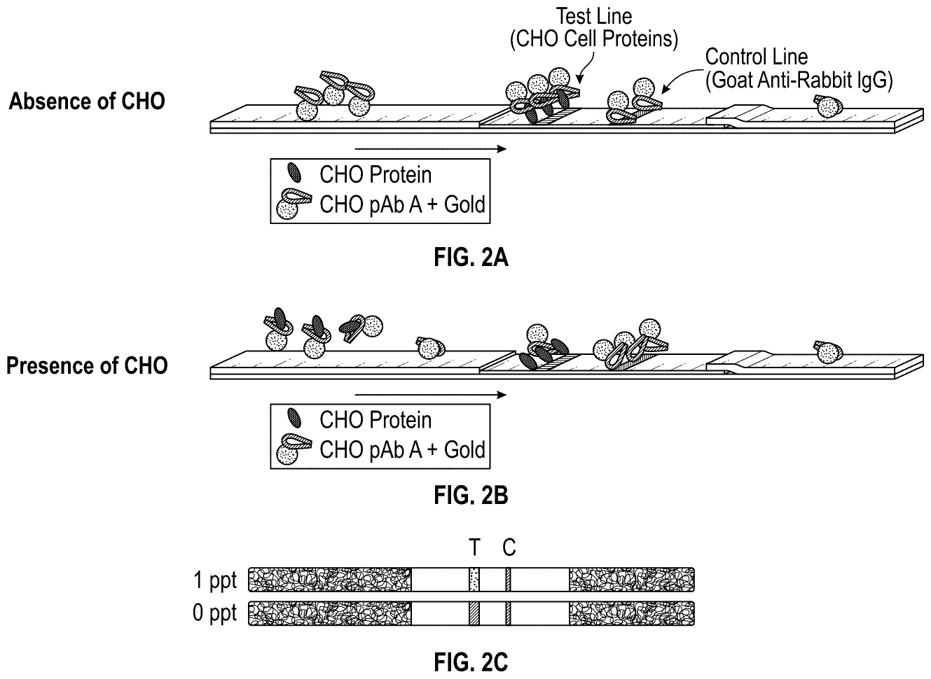

The present technology utilizes lateral flow devices that can detect the presence of impurities, such as product-related impurities, during the manufacturing process of a biopharmaceutical drug product. Lateral flow devices in accordance with the present technology can be provided in a number of different formats. FIGS. 2A and 2B illustrate detection of Chinese Hamster Ovary (CHO) cell proteins using a competitive format lateral flow device. Competitive lateral flow devices include a nitrocellulose membrane which is designed to receive or contact a prepared sample at one end. The prepared sample is a combination of a collected sample from the biopharmaceutical manufacturing process and a polyclonal antibody conjugated to a gold particle. The polyclonal antibody is a host cell line or culture polyclonal antibody and in the embodiment shown in FIGS. 2A and 2B is a CHO polyclonal antibody conjugated to a gold particle.

The competitive lateral flow device includes a detection area having a test line and in some embodiments a control line. The test line is formed from proteins or peptides, and in the embodiment shown in FIGS. 2A and 2B is formed of CHO cell proteins. The control line is formed from a common antibody (here goat anti-rabbit IgG). To detect the absence of CHO proteins in a sample collected from the biopharmaceutical manufacturing process, the sample is first collected in a vial containing a reagent (i.e., polyclonal antibody conjugated to a gold particle). To minimize time, the vial used to collect the sample can include the reagent therein. If desired, a diluent, can be added to the vial. Next, the sample receiving portion of the competitive lateral flow device is contacted to the prepared (and optionally diluted) sample. The lateral flow device can be inserted into the vial, or the prepared sample can be added dropwise onto to the receiving portion. The prepared sample then flows through the lateral flow device along the direction of the arrow shown in FIGS. 2A and 2B. As the prepared sample passes over the test line and control line binding of the gold conjugated particles occurs to provide a result, typically within two to 10 minutes from initial contact of the prepared sample with the lateral flow device. When the prepared sample is free of CHO cell proteins, the gold particles conjugated to the CHO polyclonal antibody are free to bind with the proteins in the test line and in the control line. Two bright lines indicate that the prepared sample was free of CHO proteins. However, when the prepared sample contains even a small amount (a threshold amount, e.g., 1 ppm) of CHO proteins, the gold particles conjugated to the CHO polyclonal antibody of the reagent bind during step 110, leaving less free CHO conjugated gold particles available to bind with the test line and control line. As a result, the intensity of the visual signal from the test line decreases with increasing concentration of CHO proteins within the collected sample.

FIG. 2C depicts two competitive lateral flow test strips. The sample tested using the top lateral flow strip device was spiked with 1 ppt CHO protein. The sample tested using the bottom lateral flow strip was not spiked—that is, was free of any known CHO protein. As shown in the image, the test line for the sample spiked with 1 ppt CHO protein is less intense—less visible than the control line as well as the test line on the test strip corresponding to a 0 ppt sample.

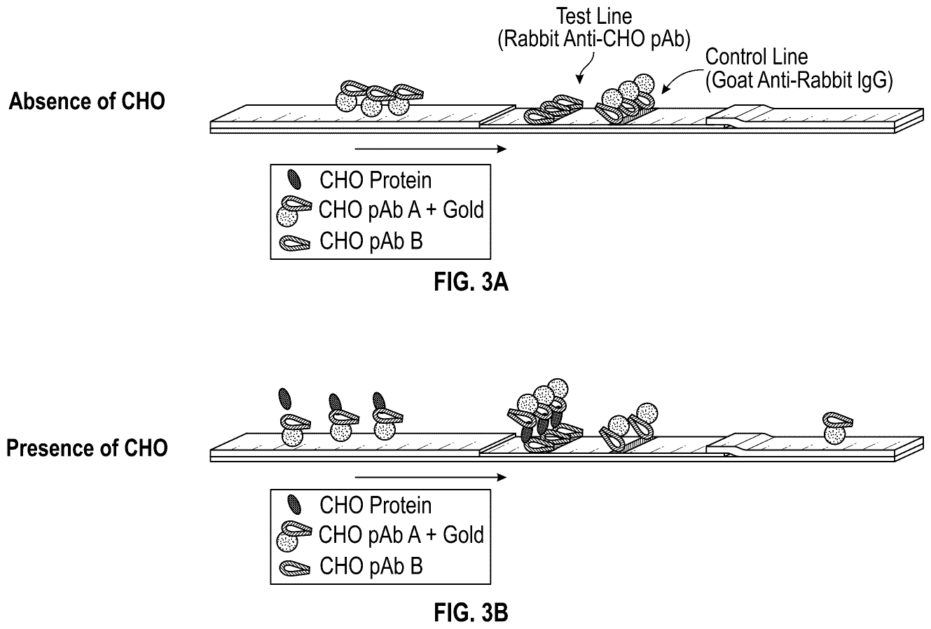

The present technology also encompasses use of sandwich format lateral flow devices. FIGS. 3A and 3B illustrate detection of Chinese Hamster Ovary (CHO) cell proteins using a sandwich format lateral flow device. Sandwich lateral flow devices also include a nitrocellulose membrane which is designed to receive or contact a prepared sample at one end. The prepared sample is a combination of a collected sample from the biopharmaceutical manufacturing process and a polyclonal antibody conjugated to a gold particle. The polyclonal antibody is a host cell line or culture polyclonal antibody and in the embodiment shown in FIGS. 3A and 3B is a CHO polyclonal antibody conjugated to a gold particle.

The sandwich lateral flow device includes a detection area having a test line and in some embodiments a control line. The test line is formed from polyclonal antibodies that specifically bind to host cell proteins from a host cell line or a host cell culture (i.e., the analyte to be detected using this lateral flow test). Typically, the polyclonal antibodies used to form the test line are different than the polyclonal antibodies conjugated to the gold particles used in the reagent. However, in some embodiments the same type of polyclonal antibodies can be used to form the test line as well as to use as a portion of the reagent. The control line is formed from a common antibody (here goat anti-rabbit IgG). To detect the absence or presence of CHO proteins in a sample collected from the biopharmaceutical manufacturing process, the sample is first collected in a vial containing a reagent (i.e., polyclonal antibody conjugated to a gold particle). To minimize time, the vial used to collect the sample can include the reagent therein. If desired, a diluent, can be added to the vial. Next, the sample receiving portion of the sandwich lateral flow device is contacted to the prepared (and optionally diluted) sample. The lateral flow device can be inserted into the vial, or the prepared sample can be added dropwise onto to the receiving portion. The prepared sample then flows through the lateral flow device along the direction of the arrow shown in FIGS. 3A and 3B.

As the prepared sample passes over the test line and control line binding of the gold conjugated particles occurs to provide a result, typically within two to 10 minutes from initial contact of the prepared sample with the lateral flow device. When the prepared sample is free of CHO cell proteins, the gold particles conjugated to the CHO polyclonal antibody of the reagent bind with the antibody in the control line. However, as there is no protein or peptide in the sample, the conjugated gold particles do not bind to the test line. The test line will be faint or absence and the presence of just a bright control line indicates that the prepared sample was free of CHO proteins. However, when the prepared sample contains even a small amount (a threshold amount, e.g., 1 ppm) of CHO proteins, the gold particles conjugated to the CHO polyclonal antibody of the reagent bind during step 110 to the CHO protein in the samples. The proteins conjugated to the polyclonal antibodies bind with the polyclonal antibodies within the test line to create a visual signal at the test line. The goat anti-rabbit IgG antibody of the control line, if included, will bind with any excess polyclonal antibody conjugated to gold particles from the prepared sample. As a result, two lines, a test line and a control line, will appear when the HCPs are present in the collected sample.

The test lines of lateral flow devices in accordance with present technology are formulated to visually illustrate the presence or absence of a threshold amount of a to be detected impurity (e.g., HCPs). In the two embodiments discussed above, the threshold amount is 1 ppt. That is, the materials deposited to form the test line are sensitive to illustrate at least a 1 ppt inclusion of the HCPs. The presence, absence, or decreased intensity of the test line provides a qualitative result, that is indicative of the absence or the presence of at least the threshold amount. Additional embodiments of lateral flow devices with different threshold amounts for the test line can also be generated. In embodiments in which a semi-quantitative result is desired, a series of lateral flow devices each having a different threshold amount associated with the test line (e.g., a first test strip with 1 ppm, a second test strip with 10 ppm, a third test strip with 50 ppm, etc.) can be utilized. By comparing the results from the series of tests taken in connection with the same sample, a semi-quantitative determination of the amount of HCPs in the collected sample can be determined.

To obtain a more accurate determination of the concentration of HCPs in the collected sample, a lateral flow reader (an apparatus that accepts lateral flow strips and detects/indicates results from a lateral flow assay) can be utilized to evaluate the intensity of the test line and control line. A non-limiting example of such a lateral flow reader includes a Vertu lateral flow reader commercially available from Vicam, Milford, MA. Typically, lateral flow readers measure the light absorbance of the test line and the control line to determine a T/C ratio. Calibration information, either contained on housing of the lateral flow device or entered into the reader is applied to determine a concentration.

The following examples illustrate various lateral flow device, reagent, and method conditions.

### Examples

### Preparation of Materials for Test Lines and Control Lines for a Competitive Lateral Flow Device

Competitive lateral flow devices were prepared to test for the presence of Chinese hamster ovary cell proteins. Materials for generating test lines (i.e., solution to be dispensed onto a nitrocellulose membrane) included 1 mg/ml of CHO lysate purchased from antibodyonline, mixed with blue dye in solution together with phosphate buffer saline (PBS) to prepare 0.5 mg/ml and 0.25 mg/ml of CHO lysate test line solution material. Control line materials were prepared using goat anti-rabbit IgG together with PBS buffer to prepare 0.15 mg/ml of control line solution. Each of the test line solution material and control solution material were dispensed using a 250 microliter syringe with a dispense rate at 1 microliter per centimeter. A discrete droplet dispenser from BioDot, Irvine, CA was used to dispense the test line and the control line on a nitrocellulose membrane to form the competitive test strip.

### Preparation of Materials for Test Lines and Control Lines for a Sandwich Lateral Flow Device

Sandwich lateral flow devices were prepared to test for the presence of Chinese hamster ovary cell proteins. Materials for generating test lines (i.e., solution to be dispensed onto a nitrocellulose membrane) included 4 mg/ml of CHO antirabbit polyclonal antibody. The polyclonal antibody was mixed with blue dye to form a 0.5 mg/ml test line solution material. Control line materials were prepared using goat anti-rabbit IgG together with PBS buffer to prepare 0.15 mg/ml of control line solution. Each of the test line solution material and control solution material were dispensed using a 250 microliter syringe with a dispense rate at 1 microliter per centimeter. A discrete droplet dispenser from BioDot, Irvine, CA was used to dispense the test line and the control line on a nitrocellulose membrane to form the competitive test strip.

### Preparation of Reagents/Polyclonal Antibodies Conjugated to Gold Particles

Three different reagents were prepared and evaluated for use. Three different sources of polyclonal anti HCP antibody were used; that is, Antibody A was used for forming reagent A, Antibody B was used for forming reagent B, and Antibody C was used for forming reagent C. Each reagent also included 60 nm gold particles procured from BBI.

Each reagent was formed as follows: 30 ml of the 60 nm gold particle solution was measured, and its pH was adjusted to 9.01 using 0.2M K₂CO₃ solution. Next the pH adjusted gold solution was split into three vials, each vial containing 10 ml. The three vials were separated. The three antibodies (Antibody A, Antibody B, and Antibody C) were diluted individually in PBS to a final concentration of 1 mg/ml. To the first of the three vials 56 microliters of antibody A solution was added; to the second of the three vials 56 microliters of antibody B solution was added; and to the third of the three vials 56 microliters of antibody C solution was added. The vials were vortexed and incubated at room temperature on rotation for two hours. The conjugate was blocked with 100 microliters of 10% BSA solution in distilled water for 1 hour; the vials were centrifuged and the pellet was resuspended in a wash buffer. The vials were centrifuged again and the pellet was then resuspended in a drying buffer to 100 milliliter. The formed reagents can be sonicated if needed and stored in a refrigerator until needed.

### Preparation of Samples for Testing Lateral Flow Strips

To test the competitive lateral flow strips, samples with known concentration of CHO protein were prepared and mixed with reagent. In this example, samples having a 0 ppt CHO protein concentration and a 1 part per thousand (ppt) concentration were prepared for lateral flow testing using the following procedure. 100 microliters of a running buffer (PBS with 2% of polysorbate surfactant (Tween 20)) were added to a 1.5 ml vial; CHO lysate at the desired concentration (0 ppt or 1 ppt) were added to the vial; 3 microliters of either reagent A (Antibody A with gold particles) or reagent B (Antibody B with gold particles) were introduced into the respective vial and mixed and then incubated at room temperature for 5 minutes. The resulting prepared samples were then ready for contact with a lateral flow strip device. Using this procedure four vials of prepared sample were created:

| | Reagent A | Reagent B |
|---|---|---|
| 0 ppt CHO Protein | Prepared sample 1 | Prepared sample 2 |
| 1 ppt CHO Protein | Prepared sample 3 | Prepared sample 4 |

### Testing Prepared Samples on Competitive Lateral Flow Strip

A dedicated competitive lateral flow strip device was placed in each of the four vials to for 5 minutes to contact with its respective prepared sample. After contact, the lateral flow strips were allowed to lay flat (i.e., in a horizontal position) and allowed to develop for 10 minutes at which time a light absorbance measurement of the test line on each of the developed test strips was read. The results were as follow:

| | Reagent A | Reagent B |
|---|---|---|
| 0 ppt CHO | 166.5 (Prepared | 251.3 (Prepared |
| Protein | sample 1) | sample 2) |
| 1 ppt CHO | 51.3 (Prepared | 99.6 (Prepared |
| Protein | sample 3) | sample 4) |

The above results show that both reagent A (which contained pAb A) and regent B (which contained pAb B) responded to CHO cell lysate at 1 ppt. These qualitative results show that a sensitivity of at least 1 ppt is possible using the methods of the present technology.

### Testing Prepared Samples on Sandwich Lateral Flow Strip

A dedicated sandwich lateral flow strip device was placed in to (1) a vial containing 0 ppt CHO Protein prepared with Reagent B and (2) a vial containing 10 ppt CHO Protein prepared with Reagent B for 5 minutes to contact with its respective prepared sample. After contact, the lateral flow strips were allowed to lay flat (i.e., in a horizontal position) and to develop for 10 minutes at which time a light absorbance measurement of the test line on each of the developed test strips was read. The results were as follow:

| | Reagent B |
|---|---|
| 0 ppt CHO | 101.1 |
| Protein | |
| 10 ppt CHO | 149.6 |
| Protein | |

The above results show that regent B (which contained pAb B) responded to CHO cell lysate at 10 ppt. These qualitative results show that a sensitivity of 10 ppt is possible using the methods of the present technology.

### Testing Prepared Samples on Competitive Lateral Flow Strip and Reading Both Test Line and Control Line

To obtain more information regarding the extent of CHO protein content in samples, test strips with both test lines and control lines were prepared using the above methods to create competitive lateral flow strip devices. In this example 6 prepared samples were created using reagent A, reagent B, and for this example reagent C.

| | Reagent A | Reagent B | Reagent C |
|---|---|---|---|
| 0 ppt CHO | Prepared | Prepared | Prepared |
| Protein | sample 1 | sample 2 | sample 5 |
| 1 ppt CHO | Prepared | Prepared | Prepared |
| Protein | sample 3 | sample 4 | sample 6 |

Six dedicated test strips (one assigned to each of the six prepared samples) were inserted into vials containing the prepared samples. After contact between the sample receiving portion of the lateral flow device with the prepared sample, the test strips were laid flat and allowed to develop for 10 minutes at which time light absorbance was read with a Vertu reader. The results are as follows:

| | CHO protein | | | |
|---|---|---|---|---|
| | concentration | | | |
| Reagent | (ppt) | T-line | C-line | T/C ratio |
| A | 0 | 495.4 | 418.3 | 1.2 |
| A | 1 | 192.5 | 514.2 | 0.4 |
| B | 0 | 339.0 | 223.5 | 1.5 |
| B | 1 | 178.1 | 349.3 | 0.5 |
| C | 0 | 623.3 | 491.1 | 1.3 |
| C | 1 | 221.5 | 660.3 | 0.3 |

The results from this data indicate that reagent C provides the greatest signal for this particular analyte.

### Testing Different Concentrations of Prepared Samples on Competitive Lateral Flow Strip and Reading Both Test Line and Control Line

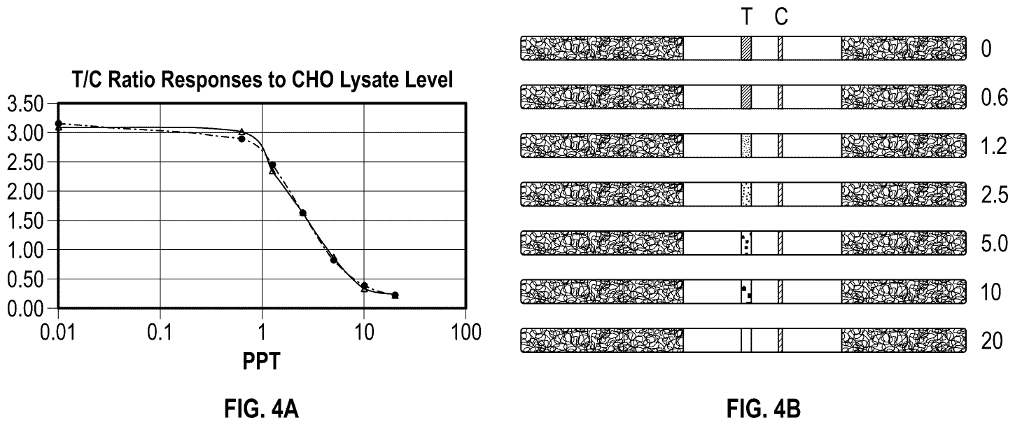

In the above examples only two concentrations of CHO protein were tested—0 ppt and 1 ppt. The results indicate that the test strips are sensitive to provide qualitative information regarding the presence or absence of CHO proteins. In this example seven different CHO concentrations were combined with reagent C to form prepared samples for competitive lateral flow testing. The seven different CHO protein concentrations were: 0 ppt; 0.6 ppt, 1.25 ppt, 2.5 ppt, 5 ppt, 10 ppt and 20 ppt. Seven vials of prepared sample using reagent C were created and dedicated test strip were inserted into the vials for 5 minutes and then laid flat to develop for 10 minutes. Light absorbance values were measured on each test line and control line. The values are shown below and plotted in FIG. 4A (solid line curve plotting data, dashed line curve plotting algorithmic model for quantitation). FIG. 4B provides an image of the series of 7 dedicated test strips.

| CHO protein (ppt) | T | C | T/C ratio |
|---|---|---|---|
| 0 | 434.6 | 140.3 | 3.1 |
| 0.6 | 508.9 | 168.8 | 3.01 |
| 1.25 | 463.5 | 197.7 | 2.34 |
| 2.5 | 349.8 | 214.4 | 1.63 |
| 5 | 235.3 | 271.3 | 0.87 |
| 10 | 94.3 | 279.8 | 0.34 |
| 20 | 67.5 | 295.2 | 0.23 |

To quantitate HCPs in the sample, each lot of strips needs to build its own standard calibration curve, which is done by testing a serial of HCPs standards at different levels (from low to high in a given range, for example from 0 to 1000 ppm). Each level of the standards will produce a T/C ratio by lateral flow strip test, and then the ratios are plotted against the concentration to generate a calibration curve. (See for example the dashed line curve in FIG. 4A). Concentration of a sample is identified by extrapolating the T/C ratio from the calibration curve.

1. A method of detecting presence of host cell proteins during production of a biopharmaceutical, the method comprising:
a) removing a liquid sample from a biopharmaceutical production reactor;
b) contacting the liquid sample to a lateral flow device, wherein the lateral flow device comprises a nitrocellulose membrane and a region of polyclonal antibodies that specifically bind to host cell proteins from a host cell line or a host cell culture; and
c) detecting presence or absence of more than a threshold concentration of host cell proteins within twenty-five minutes from removing the liquid sample from the biopharmaceutical production reactor, wherein the threshold concentration of host cell proteins is 100 ppm.

2. (canceled)

3. The method of claim 1, wherein the threshold concentration of host cell proteins is 50 ppm.

4. The method of claim 1, wherein the threshold concentration of host cell proteins is 10 ppm.

5. The method of claim 1, wherein detecting presence or absence of more than the threshold concentration of host cell proteins is within twenty minutes from removing the liquid sample from the biopharmaceutical production reactor.

6. The method of claim 1, wherein detecting presence or absence of more than the threshold concentration of host cell proteins is within fifteen minutes from removing the liquid sample from the biopharmaceutical production reactor.

7. The method of claim 1, wherein the liquid sample from the biopharmaceutical production reactor is combined with a reagent prior to contacting the liquid sample to the lateral flow device.

8. The method of claim 7, wherein the reagent comprises a buffer and gold conjugated to a polyclonal antibody of the host cell line or host cell culture.

9. The method of claim 1, wherein the reagent further comprises a diluent.

10. The method of claim 1, wherein the liquid sample from the biopharmaceutical production reactor is filtered prior to contacting the liquid sample to the lateral flow device.

11. The method of claim 1, wherein the liquid sample from the biopharmaceutical production reactor is diluted prior to contacting the liquid sample to the lateral flow device.

12. The method of claim 1, wherein the host cell line comprises Chinese hamster ovary.

13. The method of claim 1, wherein the host cell line is selected from the group of consisting of, HEK-293, HeLa, MDCK, A549, and Sf9.

14. The method of claim 1, wherein the host cell culture is selected from the group of consisting of Escherichia coli, Lactococcus lactis, Pseudomonas fluorescens, Staphylococcus aureus, Saccharomyces cerevisiae, Pichia pastoris (also known as Komagataella phaffii).

15. The method of claim 1, wherein the lateral flow device further comprises a test line and a control line.

16. The method of claim 15, wherein the method further comprises quantifying the concentration of host cell proteins present in the liquid sample via evaluating a test line light absorption level for the test line within the region of polyclonal antibodies and a control line light absorption level for the control line.

17. (canceled)

18. The method of claim 16, further comprising determining a ratio of the test line light absorption level to the control line light absorption level.

19. The method of claim 18, wherein at least a portion of the lateral flow device is inserted into a reader device for measuring a value of light absorption for the test line and the control line.

20. The method of claim 19, wherein the lateral flow device further comprises a housing, the housing having a transparent window for reading the control line and the test line, the housing further including a calibration device affixed to the housing.

21. The method of claim 1, wherein removing the liquid sample from the biopharmaceutical production reactor during a cell culturing step during production of the biopharmaceutical.

22. The method of claim 1, wherein removing the liquid sample from the biopharmaceutical production reactor is performed: prior to a purification of a cell culture within the biopharmaceutical production reactor; during a purification of a cell culture within the biopharmaceutical production reactor; or after a purification of a cell culture within the biopharmaceutical production reactor.
